(19) United States
(12) Patent Application Publication
(10) Pub. No.: US 2004/0138532 A1
(43) Pub. Date: Jul. 15, 2004

(54) Method for in vivo imaging of an unmodified gastrointestinal tract

(76) Inventor: Arkady Glukhovsky, Santa Clara, CA (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/478,252

(22) PCT Filed:
(86) PCT No.: PCT/IL02/00391

(30) Foreign Application Priority Data
May 20, 2001 (IL) 143258

(51) Int. Cl. A61B 1/06

(52) U.S. Cl. 600/176; 600/179

(57) ABSTRACT

Imaging device (40) which is capable for insertion in the intestine has a convex shaped tip (402) of endoscope (400) with an optical window (42) through which the intestine is illuminated, viewed and/or imaged.

### FIELD OF THE INVENTION

The present invention relates to in vivo imaging of the digestive tract. Specifically, the invention relates to in vivo imaging of the gastrointestinal tract in unmodified conditions.

### BACKGROUND OF THE INVENTION

Endoscopes for visual examination of body lumens usually include a flexible tube inserted into the body lumen. The tube usually includes a remote illumination source, which delivers illumination through an optical fiber, and an imaging camera, typically including a lens and an imager.

FIG. 1 schematically illustrates a prior art endoscope. In the endoscope 10, the illumination source 12 is located along side the camera 13 and camera lens 14, resulting in only partial overlapping between the field of illumination 112 and the field of view 114. Usually, the non-overlapping area between the two fields is small and not significant.

FIG. 2A schematically illustrates a prior art endoscope 20 inserted into the intestine 26 (e.g. the small intestine). The field of illumination 222 and field of view 224 overlap enabling acquisition of images from the intestine 26.

FIG. 2B schematically illustrates a prior art endoscope 20 inserted into the intestine 26 when the field of illumination 222 is obscured by a fold of the intestine 26′ or by the intestine wall collapsing on the tip of the endoscope 20. In this case there is no overlapping between the field of illumination 222 and the field of view 224 and acquisition of images of the intestine is prevented. Also, the obscuring of the field of view 224 by the fold in the intestine wall 26′ is enough to prevent image acquisition.

The problem of obscuring of the tip of the endoscope, as described above, is well known in the art and is usually solved by insufflating air in the intestine. FIG. 3 schematically illustrates a prior art endoscope 30 in an air insufflated intestine 36. Air insufflation inflates the intestinal walls, flattens the folds that are naturally present in the intestine wall, and removes potential obstruction from both the illumination source 34 (field of illumination 304) and from the lens 32 (field of view 302).

Air insufflation of the intestine, although solving problems of optical obstruction, changes the normal physiological conditions of the intestine. Under normal physiological conditions the intestine is collapsed and most of the remaining space is filed with the gastrointestinal liquid. Under insufflation the intestine is filled with air leaving the liquid spread as a moisture layer only on the intestinal wall. In an unmodified environment viewing conditions in the intestine are similar to underwater viewing. Air insufflation modifies these conditions possibly leading to degradation of some colors seen through the air (similar to tropical fishes which have vivid colors in the water but pale colors once the fish is in the air).

In addition to differences in geometry and in the physics of viewing the intestine under modified and unmodified conditions, physiological differences may develop due to insulation and the resulting air pressure in the intestine.

For example, a tamponade effect results in increased pressure on small blood vessels possibly resulting in stopping any existing bleeding. Thus, effective detection of bleeding sites in the gastrointestinal (GI) tract may be prevented. Also, villi collapse in air, diminishing the quality of obtained images, as compared to villi floating in the gastrointestinal liquid. Further, in rare cases fatal air embolism may occur as a result of insufflation endoscopy (Katgraber F, Glenewinkel F, Fischler S, Int J Legal Med 1998; 111(3) 154-6.).

### SUMMARY OF THE INVENTION

According to one embodiment of the present invention a method and device for in vivo imaging of the gastrointestinal tract in unmodified conditions, namely under natural physiological conditions, is provided. According to an embodiment of the invention obscuring of the field of view or of the field of illuminiation is prevented.

According to one embodiment, the invention is based on viewing the intestine through an optical dome, which prevents obscuring of the field of illumination or of the field of view, due to collapse or due to a fold of the intestine wall. The invention, according to one embodiment also enables to obtain in vivo images of the gastrointestinal tract having a quality that is not impaired by modifications of the intestinal environments such as by insufflation.

The method, according to one embodiment of the invention, includes the steps of: introducing into an uninsufflated intestine an imaging device for imaging the uninsufflated intestine, and obtaining images of the uninsufflated intestine. The imaging device, according to an embodiment of the invention, comprises at least one dome shaped or convex end through which the uninsufflated intestine is illuminated and viewed.

According to another embodiment of the invention there is provided a method for viewing submucosal formations in the intestine. The method, according to an embodiment of the invention, includes the steps of: introducing into an uninsufflated intestine an imaging device for imaging the uninsufflated intestine; illuminating collapsed walls of the intestine; obtaining images of the collapsed intestine walls; and obtaining from the images of the collapsed intestine wall a view of submucosal formations of the intestine. The imaging device, according to an embodiment of the invention, comprises at least one dome shaped or convex end through which the uninsufflated intestine is illuminated and viewed.

According to one embodiment the imaging device is an endoscope comprising at least one dome shaped or convex end through which the uninsufflated intestine is illuminated and viewed.

### BRIEF DESCRIPTION OF THE DRAWINGS

The present invention will be understood and appreciated more fully from the following detailed description taken in conjunction with the drawings in which:

FIG. 1 is a schematic illustration of a prior art endoscope;

FIGS. 2A and 2B are schematic illustrations of a prior art endoscope in a gastrointestinal tract (2A) and in a portion of the gastrointestinal tract having a fold (2B);

FIG. 3 is a schematic illustration of a prior art endoscope in an insufflated intestine;

FIG. 4 is a schematic illustration of an in vivo imaging device in an intestine with unmodified environment in accordance with an embodiment of the invention;

FIG. 5 is a schematic illustration comparing the illumination of a prior art endoscope in an insufflated intestine with the illumination of an in vivo imaging device according to an embodiment of the invention;

FIG. 6 is a schematic illustration comparing the optical path of a prior art endoscope in an insufflated intestine with the optical path of an in vivo imaging device according to an embodiment of the invention;

FIG. 7 schematically illustrates an angular resolution scheme for air-insufflated endoscopy and for endoscopy in accordance with an embodiment of the invention;

FIG. 8 is a more detailed, three-dimensional scheme of the schematic illustration of FIG. 7; and

FIGS. 9 and 10 present angular resolution and apparent magnification for air-insufflated endoscopy and for endoscopy in accordance with an embodiment of the invention.

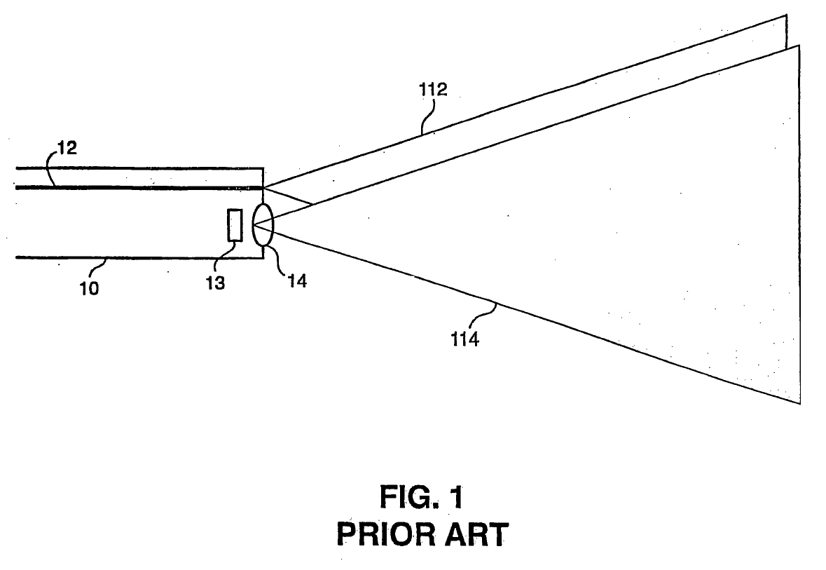

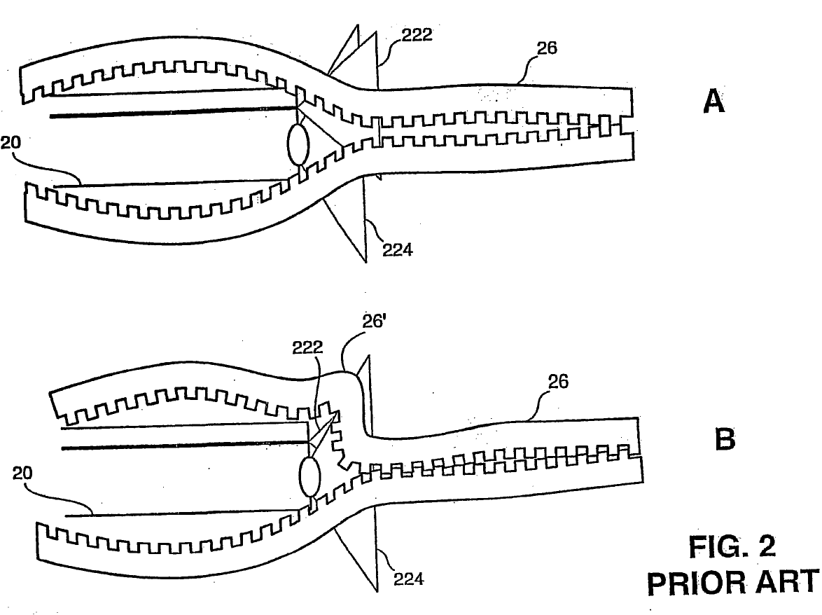

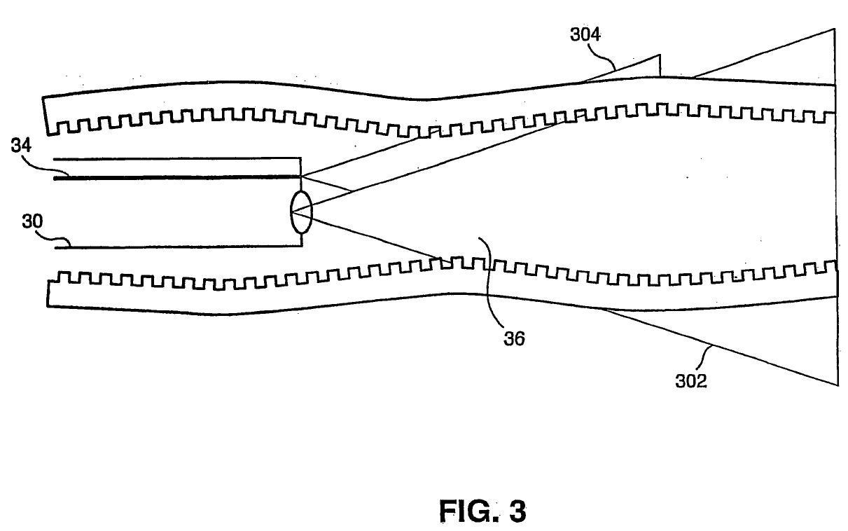

### DETAILED DESCRIPTION OF THE INVENTION

In the following discussion, embodiments of the present invention will be also referred to as “airless endoscopy”.

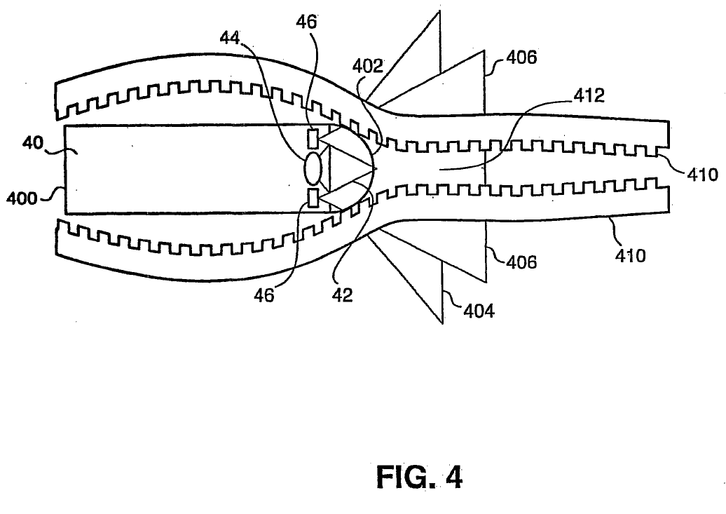

Reference is now made to FIG. 4, which presents a schematic illustration of an in vivo imaging device, specifically designed, in accordance with an embodiment of the invention, to view the gastrointestinal tract in an unmodified environment. The in vivo imaging device 40 is a device capable of being inserted and moved through the intestine, such as an endoscope. The dome or convex shaped tip 402 of endoscope 400 is an optical window 42 through which the intestine is illuminated and viewed and/or imaged. One or more illumination sources 46 and an imager and lens 44 are positioned behind optical window 42. The collapsed, uninsufflated intestine walls 410 are in close proximity to the imaging device 40 and present only a limited area 412 to be viewed. In these conditions the field of view 404 includes the entire area 412. Furthermore, illumination field (or fields) 406 provided by illumination sources 46 illuminate the entire area 412. Hence, at any point of time during the imaging device's 40 progress through the intestine a limited area of the intestine wall is fully illuminated and can be viewed in its entirety.

As will be shown and discussed below, an imaging device designed in accordance with an embodiment of the invention operates more efficiently than prior art imaging devices operating in insufflation conditions. Also, according to an embodiment of the invention it is possible to obtain images that are of an improved quality compared to images obtained under insufflation conditions. Furthermore, images obtained according to an embodiment of the invention contain information that is unobtainable under insufflation conditions of the intestine.

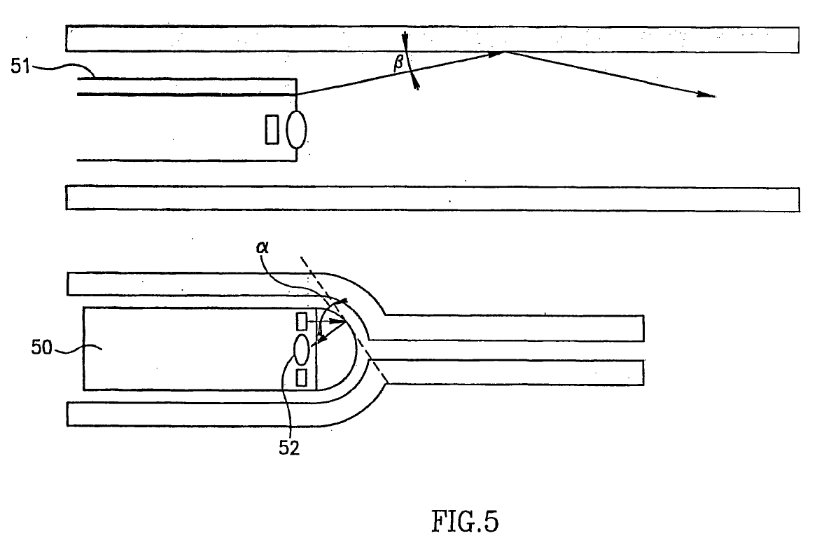

As is shown in FIG. 5 the illumination efficiency of imaging device 50, designed in accordance with an embodiment of the invention, is higher than that of prior art imaging device 51. Illumination angle α is not as sharp an angle compared with sharp illumination angle β, such that most of the illumination is efficient and is returned by the intestine wall back to the imaging device and lens 52.

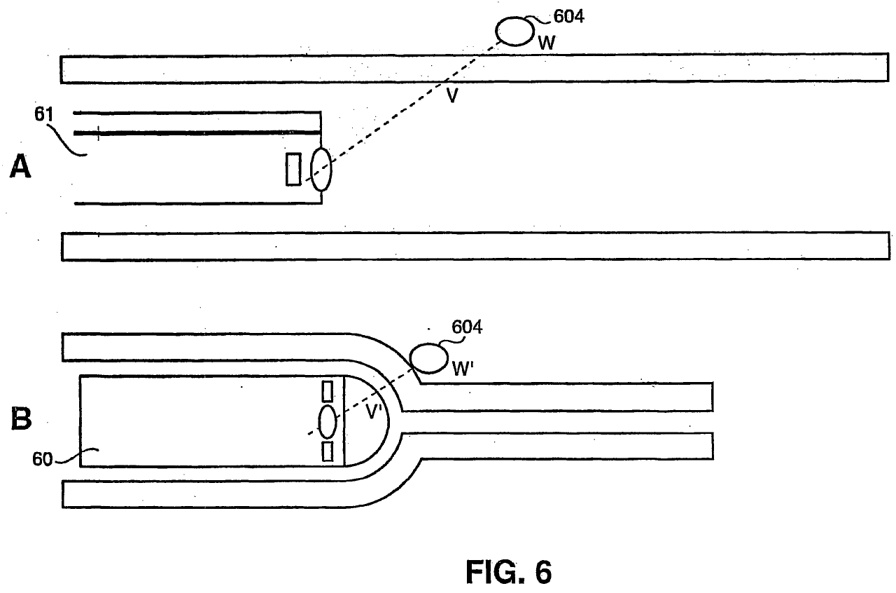

As shown in FIG. 6, also the viewing angle of the device in accordance with an embodiment of the invention is less sharp than the viewing angle of a prior art imaging device. Therefore, formations such as arterioles, venulas, lymphatic ducts and others, which are located submucosively and which are viewed according to an embodiment of the invention, are viewed through a thinner layer of mucosa than while being viewed by a prior art imaging device. As can be seen in FIG. 6, the optical path to the submucosal formation 604 is shorter (see distance v′-w′) when using an imaging device 60 in accordance with an embodiment of the invention than when using a prior art imaging device 61 (see distance v-w).

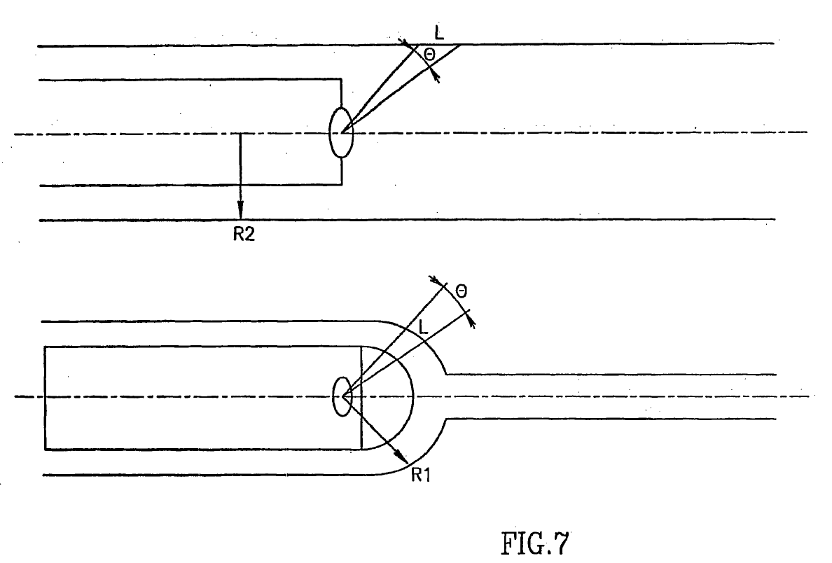

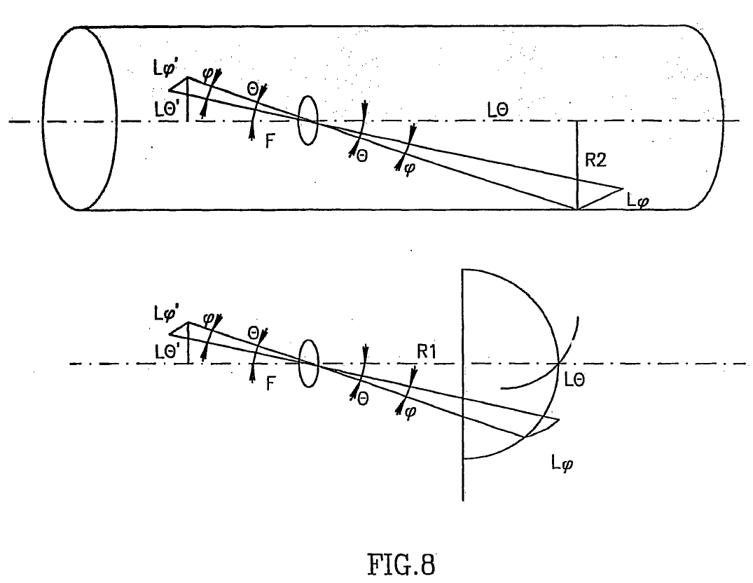

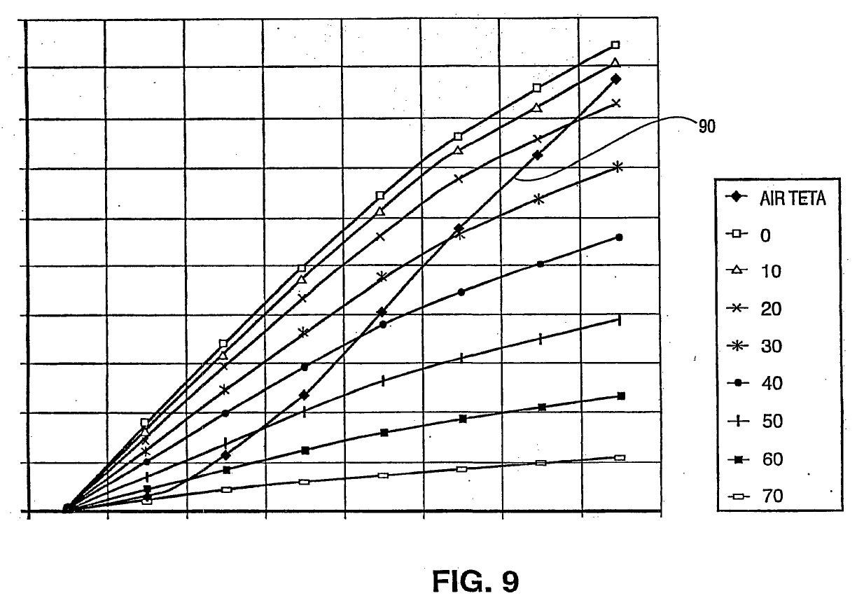

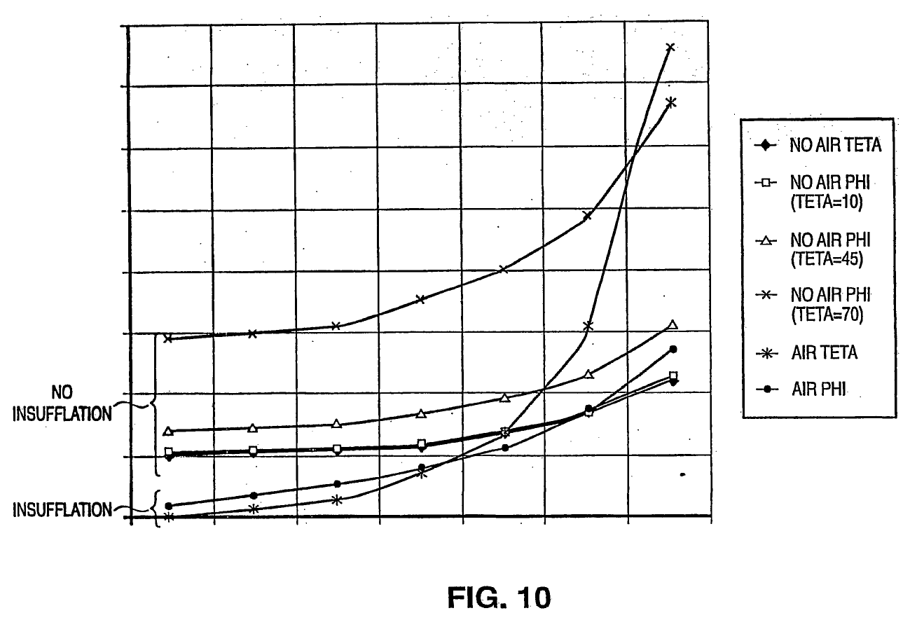

Reference is now made to FIGS. 7-10. Due to the different geometry of the two techniques (prior art compared with a method according to an embodiment of the invention), their spatial resolution is also different. FIG. 7 presents a simplified planar scheme for calculation of angular resolution and apparent magnification FIG. 8 presents a more detailed, three-dimensional scheme. FIGS. 9 and 10 present angular resolution and apparent magnification for air insufflating and airless endoscopy. It can be understood that airless endoscopy provides superior resolution in most angles of the field of view. For the simplicity of discussion several assumptions can be made: Insufflation causes the intestine to be cylindrically shaped with a radius R2. The endoscopic optical axis aligns with the geometrical axis of the intestine.

In the airless endoscopy collapsed walls of the intestine form a half-sphere around the optical dome of the endoscope. Radius half a sphere is R1. Both cases are shown in FIG. 7. It should be noted that usually R1<R2.

Finding Angular Resolution

The objective is to find what will be the angles Δθ and Δφ (along the two orthogonal axis) for an object that has length of ΔL_{θ} (or ΔL_{φ}), as a function of the view angle θ (or φ) correspondingly.

Case 1. Air Insufflating Endoscopy.

a. Angle (axis) θ

L_{θ}=R₂*ctgθ

dL_{θ}/dθ=R₂*(−1/sin²θ)

It should be noted that only an absolute value of the expression is important, therefore the minus sign will be omitted.

b. Angle (axis) φ

L_{φ}=(R₂/sin θ)*tg φ

dL_{φ}/dφ=(R₂/sin θ)*(1/cos²φ)

Case 2. Airless Endoscopy.

a. Angle (axis) θ

L_{θ}=R₁*θ

dL_{θ}/dθ=R₁

b. Angle (axis) φ

L_{φ}=R₁*φ

dL_{φ}/dφ=R₁

Assuming R1=R2 the normalized (relative) angular resolution of both prior art method and of a method according to an embodiment of the invention, may be calculated. FIG. 8 presents results of the calculation (in order to make the visual presentation more illustrative, inverse value is plotted: dθ/dL

Angular resolution of the airless endoscopy is better, especially for low values of the viewing angles.

Finding Apparent Magnification.

FIG. 9 shows geometrical relations used for calculation of linear resolution (apparent magnification), for air insufflation, and for airless endoscopy.

Magnification is defined as a ratio between the size of the object on the imager and actual size of the object. It may be defined without units or with units, e.g. [pixel/meter].

Angular resolution [m/?] versus view angle for air insufflating endoscopy. The line referred to as line 90 describes resolution versus θ. Other lines describes resolution versus φ (angle θ serves here as a parameter). Angular resolution in airless endoscopy does not depend on the viewing angle and is 1, assuming the same diameter of the intestine (R₁=R₂). Difference in the resolution is increases assuming that the insufflation increases the diameter of the intestine (R₁<R₂).

Case 1. Air Insufflating Endoscopy.

a. Angle (axis) θ

tgθ=L_{θ/F=R}₂/L_{θ}

L_{θ}=R₂/tgθ

From here:

L_{θ}=F*R₂/L_{θ}=F/tgθ

Derivative will show the linear resolution:

DL′_{θ}/dLθ=−R₂*F/L_{θ}²=−R₂*F/(R²/tgθ²)=−F/R₂*tg²θ

b. Angle (axis) φ

L′_{φ}=F/cos θ*tgφ

L_{φ}=R₂/sin θ*tg_{φ}

L′_{φ}=F/cos θ*(sin θ/R₂)*L_{φ}=F/R₂*tgθ*L_{φ}

dL′_{φ}/dL_{φ}=F/R₂*tgθ

Case 2. Airless Endoscopy.

a. Angle (axis) θ

L′_{θ}=(F*tgθ/R₁*θ)*L_{θ}

dL′_{θ}/dL_{θ}=F*tgθ/R₁*θ

b. Angle (axis) φ

L′_{φ}=(F/R₁*tgφ/φ*1/cos θ)*L_{φ}

dL′_{φ}/dL_{φ}=F/R₁*tgφ/φ*1/cos θ

Magnification for different viewing angles is shown in FIG. 10. In FIG. 10 Magnification [pixels/meter] versus view angle for air insufflating endoscopy is compared to airless endoscopy (assuming the same diameter of the intestine). For airless endoscopy magnification versus θ is shown, and magnification versus φ is shown, while θ is a parameter: θ=10° θ=45° θ=70°. For air insufflating endoscopy magnification versus θ and φ is shown. It may be seen that magnification (resolution) of airless endoscopy is superior, especially in a central area. Assuming that the insufflation increases the diameter of the intestine (R1<R2)—performance of the airless endoscopy is improved.

It may be concluded that linear resolution of airless endoscopy is superior to air insufflating endoscopy in most angles of the field of view. FIG. 10 presents the case when diameter of the intestine is the same in the air-insufflating and airless endoscopy (R1=R2). It is a reasonable assumption that the air insufflation increases the diameter of the intestine (R1>R2). Therefore linear resolution in the air-insufflating case will have even lower values than that presented in FIG. 10.

It will be appreciated by persons skilled in the art that the present invention is not limited to what has been particularly shown and described hereinabove. Rather the scope of the present invention is defined only by the claim which follow:

1. A method for in vivo imaging of an unmodified gastrointestinal tract, the method comprising the steps of:
introducing an imaging device into an unmodified gastrointestinal tract, said imaging device comprising at least one convex end through which the unmodified gastrointestinal tract is illuminated and viewed; and
obtaining images of the unmodified gastrointestinal tract.

2. The method according to claim 1 wherein the unmodified gastrointestinal tract comprises an uninsufflated gastrointestinal tract.

3. The method according to claim 1 wherein the unmodified gastrointestinal tract is an uninsufflated small intestine.

4. The method according to claim 1 wherein the imaging device is an endoscope.

5. A method for viewing submucosal formations in a gastrointestinal tract, the method comprising the steps of:
introducing an imaging device into an unmodified gastrointestinal tract, said imaging device comprising at least one convex end through which the unmodified gastrointestinal tract is illuminated and viewed;
illuminating at least one collapsed wall of the gastrointestinal tract;
obtaining images of the collapsed gastrointestinal tract wall; and
obtaining a view of submucosal formations of the gastrointestinal tract from the images of the collapsed gastrointestinal tract wall.

6. The method according to claim 5 wherein the unmodified gastrointestinal tract comprises an uninsufflated gastrointestinal tract.

7. The method according to claim 5 wherein the unmodified gastrointestinal tract is an uninsufflated small intestine.

8. The method according to claim 5 wherein the imaging device is an endoscope.

9. An endoscope configured to image an unmodified gastrointestinal tract said endoscope comprising at least one convex end through which the gastrointestinal tract is illuminated and viewed.

10. The endoscope according to claim 9 wherein the unmodified gastrointestinal tract comprises an uninsufflated gastrointestinal tract.

11. The endoscope according to claim 9 wherein the unmodified gastrointestinal tract is an uninsufflated small intestine.

12. The endoscope according to claim 9 comprising a convex optical window, an image sensor and an illumination source, wherein the image sensor and illumination source are both positioned behind said optical window.
